(19) United States
(12) Patent Application Publication
(10) Pub. No.: US 2020/0113961 A1
(43) Pub. Date: Apr. 16, 2020

(54) COMPOSITIONS COMPRISING ESSENTIAL OILS FOR TOPICAL USE

(71) Applicant: IDENA S.P.A., MILANO (IT)

(72) Inventor: Ezio BOMBARDELLI, GROPELLO CAIROLI (PV) (IT)

(21) Appl. No.: 16/603,995

(22) PCT Filed: Apr. 9, 2018
(86) PCT No.: PCT/EP2018/059015

§ 371 Date: Oct. 9, 2019

(30) Foreign Application Priority Data
Apr. 12, 2017 (IT) 102017000040834

(51) Int. Cl. A61K 36/752; A61K 36/53; A61K 36/54; A61K 31/045; A61K 31/22; A61K 31/015; A61K 9/00; A61P 3/04

(52) U.S. Cl. CPC A61K 36/752; A61K 36/53; A61K 36/54; A61P 3/04; A61K 31/22; A61K 31/015; A61K 9/0014; A61K 31/045

(57) ABSTRACT

Disclosed are compositions including one or more essential oils selected from oils deriving from citrus fruits, Lavandula officinalis or Cinnamomum cassia. The compositions may preferably be administered topically. The compositions are useful in the prevention and/or treatment of insomnia and anxiety, and in reducing appetite and stimulating the metabolism.

### TECHNICAL FIELD OF INVENTION

The present invention relates to compositions comprising one or more essential oils selected from oils deriving from citrus fruits, Lavandula officinalis or Cinnamomum cassia, which are useful in the treatment of insomnia and anxiety, and in reducing appetite and stimulating the metabolism.

### PRIOR ART

Anxiety, depression and obesity are among the most common disorders of the modern era, affecting a large proportion of the population ranging from infancy to adulthood.

Dysfunctions such as insomnia, states of anxiety and mild depression often occur as a result of stress, competitiveness and metabolic disorders, and their treatment requires the use of specific, potent medicaments which are inadvisable because of their side effects and the possible risk of dependence.

Sleep disorders have a significant incidence in the oncological and pre-operative fields, and are added to problems liable to cause a deterioration in the patient's quality of life.

Anxiety and depression are often directly or indirectly correlated with metabolic disorders, leading to problems in personal relationships.

Excess weight at pre- and post-pubertal age, and obesity in general, create serious problems of depression generated by contact with the opposite sex or inability to follow prescribed diets.

Excess weight is also one of the major health problems because it is positively correlated with an increased risk of numerous serious disorders, such as cardiovascular diseases, diabetes and cancer.

Anxiety, depression and insomnia are correlated with one another in the absence of specific psychiatric disorders.

In adults of both sexes, insomnia is a very widespread problem, which affects a growing number of individuals of working age, between 30 and 50, as a result of stress-induced anxiety, which makes the individual tired and irascible; hence the need for non-sedative tranquillisers with a calming, sleep-inducing function which act in the most physiological way possible.

Sleep, and specifically good sleep quality, is one of the essential ingredients for a good quality of life, in order to maintain the individual's physical, social, emotional and functional well-being.

Sleep is one of the main sources of an individual's energy, especially in patients suffering from serious disorders, who are already exhausted by the illness.

Insomnia is a sleep disorder characterised by inability to sleep, even though the body has a real physiological need for sleep. Insomnia can be defined as the subjective perception of difficulty with the beginning, duration and consolidation of sleep, leading to a reduction in its quality, which takes place despite sufficient opportunities for rest, and leads to impairment during the day.

This is associated with a poor quality of daytime life, involving symptoms such as fatigue, learning and memory difficulties, and lack of interest in everyday activities. The psychological impact on personal relationships is considerable, and the resulting irritability worsens the situation.

Unless otherwise indicated, “insomnia” refers to chronic insomnia, which has been present for at least a month; otherwise the disorder is known as acute or transient insomnia, which can last for periods ranging from days to weeks.

The symptoms of insomnia can be found in about 33% to 50% of the adult population; when associated with stress or weakness they are present in between 10 and 15% of cases, whereas insomnia associated with specific disorders affects between 5 and 10% of cases.

Risk factors for insomnia include aging, female sex, the presence of co-morbidities (medical, psychiatric, etc.), shift work, and the onset of devastating socio-economic situations.

The main disorders characteristic of insomnia relate to difficulty in getting to sleep, lengthy periods of waking during the night, and non-restorative or poor-quality sleep, with individual variables associated with the methods of onset, duration, frequency and perpetuation of the triggers, the type of present and past treatments used, and their efficacy.

Common problems associated with insomnia are an average sleep onset latency>30 minutes, periods of night waking with a total duration>30 minutes, and sleep duration of less than 6 hours per night.

To ensure the success of treatment, it is necessary to identify whether the patient suffers from real insomnia or a low amount of sleep due to incorrect behaviour or states relating to respiratory problems, restless leg syndrome, parasomnias, gastroesophageal reflux disease, etc.

Some individuals who suffer from hypersomnia, i.e. abnormal daytime drowsiness, complain that they are unable to fall asleep and only sleep for a few minutes, associated with agitation, which can become chronic with time.

Hypersomnia is not a common situation, whereas chronic insomnia is very frequent and alters the natural sleep cycle, which is difficult to restore, partly because sufferers try to sleep when they can during the day and then remain awake at night, thus disrupting the circadian rhythms. At this point, sleep deprivation can cause physical and mental problems which are so serious as to require corrective medicaments; however, like all drugs which act on the central nervous system, they are not free of major side effects.

The risks of long-term insomnia are cardiovascular, metabolic and immune deficiency-related. Insomnia in its various forms is a problem which has been known since ancient times. In traditional medicine, numerous plants have been identified which, taken individually or in, even complex, mixtures, seem to improve the psychosomatic state. In fact, as in the case of all symptoms involving the nervous system, the placebo effect is significant.

Extracts with psychometabolic effects can also be used to regulate the appetite and body weight, with psychosomatic repercussions.

Products which are widely used orally are those derived from plants belonging to the Papaveraceae and Valerianaceae families, which have a hypnotic effect, or families of plants containing sedative flavonoids, such as passion flower, hawthorn, chamomile or plants rich in essential oils, Lavandula, Citrus and others.

The Valerianaceae are widely used although they do not have clinically supported activities, as in the case of many other commonly used plants. Some essential oils administered orally at high doses, or as aromatherapy devised at the time of use, recently entered the pharmaceutical and nutraceutical formulary.

An essential oil of lavender was recently introduced onto the market as an anxiolytic at a dose of 80 mg/unit in capsules, and its efficacy was compared with a well-known benzodiazepine (Woelk. Phytomedicine 2010, 12, 94-99; Kasper, European Neuropsycopharmaclogy, 2015, 25, 1960-67).

However, high doses of essential oils are not free of gastroenteric side effects, in view of the considerable antibacterial activity of those active ingredients and a fairly high irritant power, and can lead, for example, to gastroesophageal reflux. In the long term, continuous oral use of large amounts of essential oils leads to problems of renal elimination, to the extent that the treatment has to be discontinued.

The classic aromatherapy treatment of anxiety and sleep disorders is known in traditional medicine, wherein its use is left to the user, who applies the oil to various parts of the body, such as the temples or feet, or sprinkles a few drops in boiling water and inhales the vapours. However, these uses are not standardisable, and in particular are not easy to apply; for these reasons, the approaches used in traditional medicine have not led to definite conclusions about their efficacy and use. Oral administrations at high doses were introduced to eliminate these drawbacks, but this involves the adverse effects described above.

Navarra M. et al., Frontiers in Pharmacology, vol.6, 02 March 2015, article 36, disclose the use of Citrus bergamia essential oil in aromatherapy and mentions its ability to improve mood and the mild symptoms of stress disorders.

Link, V. M. et al., Phytomedicine, 16 (2009) 303-307 disclose the use of linalool-induced sedation in mice when inhaled by vaporization.

De Sousa, D. P. et al, Molecules 2015, 20, 18620-18660 disclose the sedative and anxiolytic activity of linalool and limonene when administered by inhalation.

Cheng, Bing-Ho et al., Journal of Traditional and Complementary Medicine, vol. 5, n. 1 (2014), pages 27-34 disclose the anxiolytic activity of linalool from Cinnamomum osmopholeum when orally administered to mice.

Katsuya N. et al., Autonomic neuroscience: Basic and Clinical 185 (2014) 29-35 review the effects of the olfactory stimulation with grapefruit and lavender oil on autonomic nerve activity and physiological function and their effect in the reduction of appetite.

However, none of the above documents specifically teaches the use of essential oils in compositions for nasal, perinasal or labial administration.

CN 106266963A discloses the use of cinnamon and Citrus aurantium in a composition in the form of a lotion, an ointment or a powder for the treatment of fatty liver and hyperlipidemia. This document is silent on essential oils and on the use of the same for the treatment of anxiety or for the reduction of appetite.

There is consequently still a need to identify alternative products useful in the prevention and/or treatment of insomnia and anxiety, and in appetite reduction and metabolic stimulation.

### SUMMARY OF THE INVENTION

The present invention relates to compositions comprising one or more essential oils deriving from citrus fruits, Lavandula officinalis or Cinnamomum cassia.

The invention also relates to the use of the compositions in the prevention and/or treatment of insomnia and anxiety, in appetite reduction and metabolic stimulation, in treatment of excess weight and obesity, and in body weight reduction.

### DETAILED DESCRIPTION OF THE INVENTION

It has surprisingly been found that compositions comprising one or more essential oils selected from oils deriving from citrus fruits, Lavandula officinalis or Cinnamomum cassia are useful in the treatment of insomnia and anxiety, appetite reduction and metabolic stimulation.

The essential oils are preferably obtained from the fruit of Citrus bergamia, Citrus limon, Citrus paradisi and Lavandula officinalis. Said oils may be obtained traditionally by steam current distillation or extraction with hypercritical gases. The essential oil is preferably obtained from Citrus bergamia.

Essential oil of Citrus bergamia can be prepared by pressing, and preferably contains 35% linalool, 30% linalyl acetate and 20% limonene, together with small amounts of other terpenes.

Essential oil of Citrus paradisi may be prepared by pressing and distillation.

Essential oil of Lavandula officinalis preferably contains linalool and linalyl acetate in about the same ratio, together with a small portion of other monoterpenes.

Said compositions can contain a single essential oil or mixtures of said essential oils.

Alternatively, the compositions may comprise the active ingredients contained in said essential oils, such as linalool, linalyl acetate and limonene. For example, the compositions can comprise linalool, linalyl acetate and limonene, preferably in a weight ratio of 1:1:1.

According to a preferred aspect, the compositions comprise as essential oil only an essential oil of Citrus bergamia.

According to another preferred aspect, the compositions comprise an essential oil of Citrus bergamia combined with essential oil of Lavandula officinalis, preferably in a weight ratio of 10:1.

According to a further preferred aspect, the compositions comprise as essential oil only an essential oil of Citrus paradisi.

According to a still further preferred aspect, the compositions comprise an essential oil of Citrus paradisi and an essential oil of Cinnamomum cassia.

In another further preferred aspect, the compositions comprise an essential oil deriving from citrus fruit, an essential oil of Lavandula officinalis and an essential oil of Cinnamomum cassia.

In the compositions according to the invention, the one or more essential oil , may range from 2% to 25% w/w, and preferably amounts to 15% or 20% w/w with respect to the overall weight of the composition.

In compositions wherein Citrus bergamia essential oil is the sole essential oil, its amount may range from 5% to 25% w/w, and preferably amounts to 10% or 15% w/w.

In compositions wherein Citrus paradisi (grapefruit) essential oil the sole essential oil, its amount may range from 10% to 25% w/w, and preferably amounts to 18% or 20% w/w.

The compositions according to the invention may be administered one to three times a day, preferably applying 10-50 mg of composition in the nasal and perinasal area.

According to a further aspect, the compositions may be administered topically, for example nasally, perinasally, labially, or in other suitable parts of body.

The compositions may be incorporated in semisolid formulations, such as ointments, gels or creams. Such formulations include, in addition to the one or more aforementioned essential oils, other ingredients or excipients suitable to dilute the oil(s) to a desired concentration and to allow topical application. Such other ingredients include vegetable oils and/or fats like, for example, Ricinus communis seed oil, avocado oil, Prunus amygdalus dulcis oil, shea butter; thickening agents; surfactants and antioxidants.

The compositions according to the invention may be formulated according to conventional techniques, such as those described in Remington's Pharmaceutical Handbook, Mack Publishing Co., N.Y., USA.

The compositions according to the invention are useful in the treatment of insomnia and anxiety, appetite reduction and metabolic stimulation.

The compositions according to the invention, reducing appetite and/or stimulating metabolism, are consequently useful in weight reduction in general and, in particular, may be used in the treatment of excess weight and obesity.

It has surprisingly been found that the compositions according to the invention, administered topically in the labial, perinasal and nasal areas, induce sleep in individuals suffering from insomnia, which is often anxiety-dependent, in a short time and a comfortable way, without any side effects in view of the small amount of essential oils administered.

Moreover, the compositions are characterised by ease of use, even during the night when the patient wakes up and has difficulty getting back to sleep again.

The compositions according to the present invention are particularly useful in paediatrics, as the waking/sleep problem is particularly significant in growing children.

Essential oils are also characterised by an analgesic activity which can be useful in states of anxiety and insomnia, especially when associated with chronic pain in the elderly and in long-term oncological patients, before moving on to appropriate painkilling treatments. The target of the active ingredients is mainly the limbic system of the hypothalamus, from which biochemical and neuronal messages are transmitted to specific sites. The hypothalamic-pituitary-adrenal axis is involved in metabolic regulation as well as insomnia.

The combinations to be administered to humans were tested on a preliminary basis on rats, monitoring the electroencephalogram before and during the treatment and placing the laboratory animals in a controlled atmosphere of active ingredient, as reported in the literature to evaluate its efficacy in aromatherapy.

Accordingly, the present invention further relates to a method for treating insomnia and anxiety, in particular anxiety-related insomnia, said method comprising administering to a subject in need thereof an effective amount of a composition, preferably in the form of an ointment, gel or cream, said composition comprising one or more essential oils selected from oils deriving from citrus fruits, Lavandula officinalis or Cinnamomum cassia.

In a preferred aspect, the method is a method for treating anxiety-related insomnia, said method comprising administering to a subject in need thereof an effective amount of a composition comprising as essential oil only an essential oil of Citrus bergamia. Preferably, the composition comprises also Ricinus communis seed oil, avocado oil and Prunus amygdalus dulcis oil.

The present invention also relates to a method for reducing appetite and stimulating metabolism, for the treatment of excess weight and obesity and for body weight reduction said method comprising administering to a subject in need thereof an effective amount of a composition, preferably in the form of an ointment, gel or cream, said composition comprising one or more essential oils selected from oils deriving from citrus fruits, Lavandula officinalis or Cinnamomum cassia.

In a preferred aspect, the method is a method for reducing appetite, stimulating metabolism and reducing body weight, said method comprising administering to a subject in need thereof an effective amount of a composition comprising as essential oil only an essential oil of Citrus paradisi. Preferably, the composition further comprises also Ricinus communis seed oil, avocado oil and Prunus amygdalus dulcis oil.

The examples below further illustrate the invention.

### Example 1-Formulation as a Perinasal Ointment Based on Essential Oil of Citrus bergamia

A composition was prepared comprising (% w/w):

| | |
|---|---|
| Essential oil of Citrus bergamia | 15% |
| Ricinus communis seed oil | 20% |
| Avocado oil | 25% |
| Prunus amygdalus dulcis oil | 15% |
| Dermofeel ® Viscolid | 5% |
| Dermofeel ® Sensolv | 10% |
| Shea butter | 9.5% |
| Aperoxid ® TLA | 0.5% |

### Example 2

A composition was prepared comprising (% w/w):

| | |
|---|---|
| Essential oil of Citrus bergamia | 10% |
| Essential oil of Lavandula officinalis | 5% |
| Ricinus communis seed oil | 20% |
| Avocado oil | 25% |
| Prunus amygdalus dulcis oil | 15% |
| Dermofeel ® Viscolid | 5% |
| Dermofeel ® Sensolv | 10% |
| Shea butter | 9.5% |
| Aperoxid ® TLA | 0.5% |

### Example 3

A composition was prepared comprising (% w/w):

| | |
|---|---|
| Essential oil of Citrus paradisi | 20% |
| Ricinus communis seed oil | 20% |
| Avocado oil | 25% |
| Prunus amygdalus dulcis oil | 10% |
| Dermofeel ® Viscolid | 5% |
| Dermofeel ® Sensolv | 10% |
| Shea butter | 10% |

### Example 4

A composition was prepared comprising (% w/w):

| | |
|---|---|
| Essential oil of Citrus paradisi | 18% |
| Essential oil of Cinnamomum cassia | 2% |
| Ricinus communis seed oil | 20% |
| Avocado oil | 25% |
| Prunus amygdalus dulcis oil | 10% |
| Dermofeel ® Viscolid | 5% |
| Dermofeel ® Sensolv | 10% |
| Shea butter | 10% |

### Example 5

A composition was prepared comprising (% w/w):

| | |
|---|---|
| Linalool | 6% |
| Linalyl acetate | 8% |
| Limonene | 6% |
| Ricinus communis seed oil | 20% |
| Avocado oil | 25% |
| Prunus amygdalus dulcis oil | 10% |
| Dermofeel ® Viscolid | 5% |
| Dermofeel ® Sensolv | 10% |
| Shea butter | 10% |

### Example 6-Efficacy Test

The efficacy of the compositions according to the invention in clinical pharmacology was evaluated by examining parameters relating to induced anxiety, depression and humoral mood parameters, evaluating the amount of circulating cortisol measured in the saliva by a known clinical procedure.

In the modulation of anxiety-related insomnia, 36 female patients suffering from insomnia as a result of anxiety states of various origins were treated. The patients randomised with a comparable neurovegetative profile were treated with a placebo (formulation without essential oil) or with the formulation of example 1, administered in the labial, perinasal or proximal nasal area for 2 weeks. They were required to take the formulation before going to bed, preferably always at the same time. In the morning, each patient was asked to fill in a questionnaire reporting on the speed of falling asleep, any calming effect following the treatment, sleep quality, and in the event of nocturnal threshold consciousness, its frequency. The evaluation criterion used was the Leeds Sleep Evaluation Questionnaire (SEQ), a tool designed to evaluate the effects of psychoactive drugs on sleep, which consists of a self-assessment with a visual analogue technique. The SEQ explores 10 aspects of sleep, including the following four:

Getting to sleep (GTS),

Quality of sleep (QOS),

Awakening from sleep (AFS),

Behaviour following wakefulness (BFW).

Treatment with the formulation of example 1 produced a mediated global difference of 35% compared with the placebo, and a difference of 72±21% compared with the baseline value.

According to the statistical analysis, the efficacy was much higher for the composition of example 1 than the placebo. Example 7-Efficacy Test

In the case of appetite modulation and metabolic stimulation, the procedure described below was followed.

The patients were treated during the day with the same amount of formulation taken 3 times a day at mealtimes (breakfast, lunch and dinner). The cortisol content of the saliva was evaluated 15 min. after administration to provide biochemical evidence of the ingestion of the product.

For appetite control in overweight patients with a BMI>30 following a forced low-calorie diet, Citrus paradisi oil was administered, and the patients were monitored with the Haber scale.

Clinically, the formulation of example 3 was administered to 42 patients, mainly women, in a double-blind trial; after randomisation the patients were divided into two groups and treated with the placebo or the formulation of example 3 for 60 days, applying the formulation perinasally 10 minutes before the main meals. For the efficacy evaluation in terms of satiety and consequently of appetite, the Haber scale was used, which has appetite as primary endpoint and psychosomatic parameters as secondary endpoints. The Haber scale is a visual scale scored from −10 to +10, wherein −10 represents maximum hunger and +10 represents maximum satiety. The patient was asked to indicate their sensation of hunger before the midday meal.

To ensure optimum identification of each change of satiety according to the Haber scale, the monitoring times were established equally for both groups, and the non-parametric Friedman test was used for the statistical evaluation, evaluating the sensation of satiety between the baseline and the six periods of time established for each group. The average in the Haber test was significantly higher in the group treated with the formulation of example 3 (Friedman's test 41.49 p<0.0001), shifting from negative to positive values. Specifically, the scores expressed for the sensation of satiety were considerably different from the baseline scores on each subsequent check. Conversely, the placebo group (Friedman's test 9.62 p=0.14) exhibited a weak but not significant initial increase.

The formulation of example 3 is therefore clearly superior to the placebo in terms of efficacy. Appetite reduction and body metabolism stimulation using a composition of example 3 was reflected by a reduction in body weight.

1. Compositions comprising one or more essential oils deriving from citrus fruits, Lavandula officinalis or Cinnamomum cassia.

2. Compositions according to claim 1, comprising an essential oil of Citrus bergamia in combination with an essential oil of Lavandula officinalis.

3. Compositions according to claim 2, wherein the essential oil of Citrus bergamia and the essential oil of Lavandula officinalis are in a weight ratio of 10:1.

4. Compositions according to claim 1, comprising an essential oil of Citrus paradisi and an essential oil of Cinnamomum cassia.

5. Compositions according to claim 1, wherein the essential oil is replaced with one or more active ingredients contained in the essential oil selected from linalool, linalyl acetate and/or limonene.

6. Compositions according to claim 1, wherein said compositions are administered by the topical route.

7. Compositions according to claim 6, wherein the compositions are applied in the nasal and/or perinasal and/or labial area.

8. A method for treatment of insomnia, comprising administering an effective amount of the composition of claim 1.

9. A method for treatment of anxiety, comprising administering an effective amount of the composition of claim 1.

10. A method for reduction of appetite and/or in the stimulation of the metabolism, comprising administering an effective amount of the composition of claim 1.

11. A method for treatment of excess weight and obesity, comprising administering an effective amount of the composition of claim 1.

12. (canceled).

13. Compositions according to claim 2, wherein the essential oil is replaced with one or more active ingredients contained in the essential oil selected from linalool, linalyl acetate and/or limonene.

14. Compositions according to claim 3, wherein the essential oil is replaced with one or more active ingredients contained in the essential oil selected from linalool, linalyl acetate and/or limonene.

15. Compositions according to claim 4, wherein the essential oil is replaced with one or more active ingredients contained in the essential oil selected from linalool, linalyl acetate and/or limonene.

16. Compositions according to claim 2, wherein said compositions are administered by the topical route.

17. Compositions according to claim 3, wherein said compositions are administered by the topical route.

18. Compositions according to claim 4, wherein said compositions are administered by the topical route.

19. Compositions according to claim 5, wherein said compositions are administered by the topical route.

20. A method of treating insomnia, comprising administering an effective amount of the composition according to claim 2.

21. A method of treating insomnia, comprising administering an effective amount of the composition according to claim 3.
